SEQUENCE LISTING (2 sequences)

SEQ ID NO: 1 - DNA, 675 bp, Bacteriophage
atgatggaaatcagagaaatgctagtagacccaagtaaatatggaattaaatgtccgaataaaatggcaccgaaatatattacgttccacaatacgtataacgatgctcccgcagaaaatgaggttcgttacatgatcgggaacaataacgaggtttcgttccacgttgctgtggatgataaggaagctgttcagggcattccttttgatcgaaatgcctggcattgtggagacgggaacgggacaggaaaccgtcaatccatcggcgtagagatttgttattccaagtccggcggtaaccgatattataaggccgaggacaatgcggctattatcattgcccagcttatgaaacagttttgtattcctattgagaatgtggttccacaccagcactggagtggtaaatactgtccgcacagaatgttagatgagggaagagtgccaagctttatagagcgaattaaacaagcatacgaaggagaggaagacgacatgagtagaacattacaactggaagattggcaatggaaacagctctatgacaatatggggaaagtctggaatgcagggaaatttacagactggaattggatggttaaaatagaaaaccgttgccttaccgttgatgagctggcatggcttaacaaccacattttagcgagtagcctgtag

SEQ ID NO: 2 - protein, 224 aa, Bacteriophage
MMEIREMLVDPSKYGIKCPNKMAPKYITFHNTYNDAPAENEVRYMIGNNNEVSFHVAVDDKEAVQGIPFDRNAWHCGDGNGTGNRQSIGVEICYSKSGGNRYYKAEDNAAIIIAQLMKQFCIPIENVVPHQHWSGKYCPHRMLDEGRVPSFIERIKQAYEGEEDDMSRTLQLEDWQWKQLYDNMGKVWNAGKFTDWNWMVKIENRCLTVDELAWLNNHILASSL

(19) United States
(12) Patent Application Publication
(10) Pub. No.: US 2017/0183642 A1
(43) Pub. Date: Jun. 29, 2017

(54) PAENIBACILLUS LARVAE TREATMENT WITH PHAGE LYSIN FOR AMERICAN FOULBROOD DISEASE

(71) Applicant: The Board of Regents of the Nevada System of Higher Education on Behalf of the University of Ne, Las Vegas, NV (US)

(72) Inventors: Penny S. Amy, Henderson, NV (US); Lucy LeBlanc, North Las Vegas, NV (US)

(21) Appl. No.: 15/300,693

(22) PCT Filed: Apr. 3, 2015
(86) PCT No.: PCT/US2015/024219

§ 371 Date: Sep. 29, 2016

Related U.S. Application Data
(60) Provisional application No. 61/974,667, filed on Apr. 3, 2014.

(51) Int. Cl. C12N 9/36; A61K 38/47

(52) U.S. Cl. CPC C12N 9/2462; C12Y 302/01017; A61K 38/47

(57) ABSTRACT

Materials and methods for treating and preventing American Foulbrood disease in honeybees, such as materials and methods for using phage lysin enzymes to lyse Paenibacillus larvae, are provided herein.

### CROSS-REFERENCE TO RELATED APPLICATIONS

This application claims benefit of priority from U.S. Provisional Application Ser. No. 61/974,667, filed Apr. 3, 2014, which is incorporated herein by reference in its entirety.

### STATEMENT AS TO FEDERALLY SPONSORED RESEARCH

This invention was made with government support under 2011-67013-30169, awarded by the United States Department of Agriculture/National Institute of Food and Agriculture. The government has certain rights in the invention.

### TECHNICAL FIELD

This document relates to materials and methods for treating and preventing American Foulbrood disease in honeybees, and more particularly to materials and methods for using phage lysin enzymes to lyse Paenibacillus larvae.

### BACKGROUND

Honeybees pollinate agricultural crops and native plant species around the world. Without the efforts of the bees, many food supplies would suffer. The use of industrially imported and transported bees is not a trivial endeavor. Some large bee pollination companies have a million or more hives. Such operations may truck hundreds of thousands of bee hives across the United States, e.g., to California to pollinate the almond crop grown each year. These same hives are then trucked back across the country to pollinate blueberries and other crops that bloom later than almonds. Some people make their living from harvesting honey from their bee hives. Many bee hives are kept by amateur bee keepers who enjoy the hobby and inadvertently help neighbors through the work of their bees.

An aggressive loss of bee hives has begun to devastate the world's bee population. The loss is called Colony Collapse Disorder, and its entire cause is not known. Some believe it is due to systemic pesticides used on large monoculture agricultural crops. In addition to outright death of the hives, Colony Collapse Disorder causes hives to be weakened and made vulnerable to a number of infections.

A long known infection suffered by bees is caused by the bacterium, Paenibacillus larvae. While the associated disease is called American Foulbrood disease (AFB), it is found worldwide. Infection with P. larvae is a serious disease of honeybees that eventually destroys the infected hive and further infects other hives. AFB affects the earliest stages of the larval development, just after the eggs are hatched. The young larvae are digested from the inside out by the bacteria. With the loss of the brood, the colony has no chance to recover.

Various treatments have been used for AFB, including antibiotics such as Oxytetracycline HCl and Tylosin tetrate. The bacteria quickly became resistant to the antibiotics, however, and residue from the chemicals has been found in honey. Thus, such treatment is not acceptable to the public. Additionally, the introduction of antibiotics into the environment can have serious secondary effects, such as causing other bacteria to develop general resistance to antibiotics.

The primary current treatment for the presence of P. larvae is burning of the hives, the bees, and the equipment used to support the beekeeping of that hive. State departments of agriculture have inspectors who test for the presence of P. larvae, and the treatment typically is done quickly. This is a drastic treatment, however, and the industry has been hesitant to impose regulations on the inspection and treatment of hives, or to provide any other meaningful regulations to find and address infections.

### SUMMARY

This document is based in part on the discovery of a novel phage lysin enzyme that causes the P. larvae cell wall to break open. As described herein, the gene encoding the lysin was sequenced, and the coding sequence was over-expressed using an inducible promoter. The expressed protein was collected for semi-purification, and was used to lyse P. larvae in culture. The lysin worked without the need for phage attachment to bacteria, without the need for replication, without the host bacterium for expression, and from the outside of the cell. In addition, the lysin was specific for P. larvae cells, and did not lyse other bacterial cell types, animal cells, or plant cells.

In one aspect, this document features a method for killing a P. larvae cell. The method can include contacting the cell with a lysin polypeptide having an amino acid sequence with at least 90 percent sequence identity to SEQ ID NO:2. The P. larvae cell can be present in a honeybee larva. The contacting can include providing the lysin polypeptide to the environment of a honeybee hive. The lysin polypeptide can have an amino acid sequence with at least 95 percent sequence identity to SEQ ID NO:2.

In another aspect, this document features a purified polypeptide having an amino acid sequence that is at least 90 percent but less than 100 percent (e.g., at least 95 percent but less than 100 percent) identical to the sequence set forth in SEQ ID NO:2. This document also features a composition containing the polypeptide. The composition also can contain, for example, honeybee food (e.g., a nutritional medium such as worker jelly or royal jelly).

In another aspect, this document features an isolated nucleic acid having a nucleotide sequence that is at least 90 percent but less than 100 percent (e.g., at least 95 percent but less than 100 percent) identical to the sequence set forth in SEQ ID NO:1. In addition, this document features an expression vector containing the nucleic acid.

This document also features a method for treating American foulbrood in a population of honeybees. The method can include administering to the population a lysin polypeptide having an amino acid sequence with at least 90 percent sequence identity to SEQ ID NO:2. The population can include honeybee larvae. The administering can include providing the lysin protein to the environment of a honeybee hive, providing a composition that comprises the lysin protein to the environment of a honeybee hive, or providing a nucleic acid encoding the lysin protein to the environment of a honeybee hive. The lysin polypeptide can have an amino acid sequence with at least 95 percent sequence identity to SEQ ID NO:2.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows the structure of a bacteriophage.

FIG. 2 is a graphic depicting replication of a bacteriophage during a lytic cycle.

FIG. 3 is a graphic that more completely depicts the replication stage during the bacteriophage lytic cycle.

FIG. 4 is a graphic representation of a complete lysogenic cycle for a bacteriophage.

FIG. 5 is a graphic representation of a lysin system.

FIGS. 6A and 6B depict the structure of the protein (XIII_gp38) encoded by the gene immediately downstream from the PlyPalA gene, as predicted by Phobius (FIG. 6A) and TMHMM (FIG. 6B). Both programs predicted two transmembrane domains and cytoplasmic termini for XIII_gp38, strongly suggesting that it is a class II phage holin.

FIGS. 7A and 7B show the three-dimensional structure of the PlyPalA protein, as predicted by Phyre2 (FIG. 7A) and RaptorX (FIG. 7B). The N-terminus is depicted on top and the C-terminus is depicted at the bottom of FIGS. 7A and 7B.

FIG. 8 is a diagram showing a comparative analysis of the genomes of Phage phiIBB_PI23 (top), Phage F (middle), and Phage XIII (bottom). Hatched bars indicate homologous lysin genes and bars with vertical striping indicate homologous holin genes. The level of shading indicates conservation among the genomes, with the lightest shading indicating conservation among all 3 genomes, and the darker shading indicating conservation among 2 of 3 genomes.

FIG. 9 is a graph depicting the percentages of putative catalytic activities in lysins from annotated P. larvae phages.

FIGS. 10A and 10B are pictures of gels containing PCR products after Phusion amplification of plypalA from phage XIII (FIG. 10A) and plypalB from phage F (FIG. 10B).

FIG. 11 is a picture of an SDS-PAGE showing progressive purification of PlyPalA. M, 10-250 kDa ladder from New England Biolabs; 1, AEC (DEAE) 600 mM NaCl eluate; 2, CEC (CM) flowthrough; 3, dialysate after salting out; 4, crude E. coli lysate.

FIG. 12 is a graph plotting effective percent drop in OD₆₀₀ for P. larvae strain 748 after treatment with of purified lysin (upper line) or buffer (lower line). Error bars indicate standard deviations for triplicates.

### DETAILED DESCRIPTION

P. larvae (previously classified as Bacillus larvae) is a pathogen of the larval honeybee (Apis mellifera L.), which causes American foulbrood. Bacteriophage (also called “phage”) are viruses that can infect and kill bacteria. A bacteriophage of B. larvae was first isolated from decaying larvae of bees killed by AFB (Smirnova, 1953). A second phage was isolated from a lysogenic culture of P. larvae (Gochnauer, 1955). The second phage differed from the first phage in its ability to pass through asbestos filters, heat stability, and plaque morphology (Smirnova, 1954; Gochnauer, 1970). In addition, evidence suggested that other phages were present in other strains of B. larvae (Gochnauer and L'Arrivee, 1969). This conclusion was drawn from sensitivity tests using culture filtrates from different B. larvae cultures and lawns of many different strains. No efforts were made to isolate the different phages. Researchers were unable to concentrate or purify the phage, and, thus were unable to observe the morphology or analyze the nucleic acid component of the phage.

Another phage specific for B. larvae was isolated from a soil sample in Bulgaria (Popova et al., 1976; Valerianov et al., 1976). This phage, named L3, lysed 10 of 15 strains of B. larvae tested, and did not lyse B. cereus or B. anthracis. A phage, termed BLA, was isolated in Czechoslovakia from several B. larvae strains obtained from combs containing bee larvae killed by American foulbrood (Drobnikova and Ludvik, 1982). All of the phage preparations from different cultures were typically considered to be identical, based on the sole criterion of their appearance in electron micrographs, although some researches believed that two distinct phages were present.

P. larvae in honeybees can be lysed by introducing phage into a bee hive, such that the phage can physically associate with and lyse the P. larvae. Some phage can infect multiple P. larvae strains tested, including both environmental and isolated (wild) P. larvae strains. See, e.g., U.S. Ser. No. 14/162,638, which published as US 2014-0213144. Thus, viruses are a potential means to control the bacterium and, thereby, treat P. larvae infection.

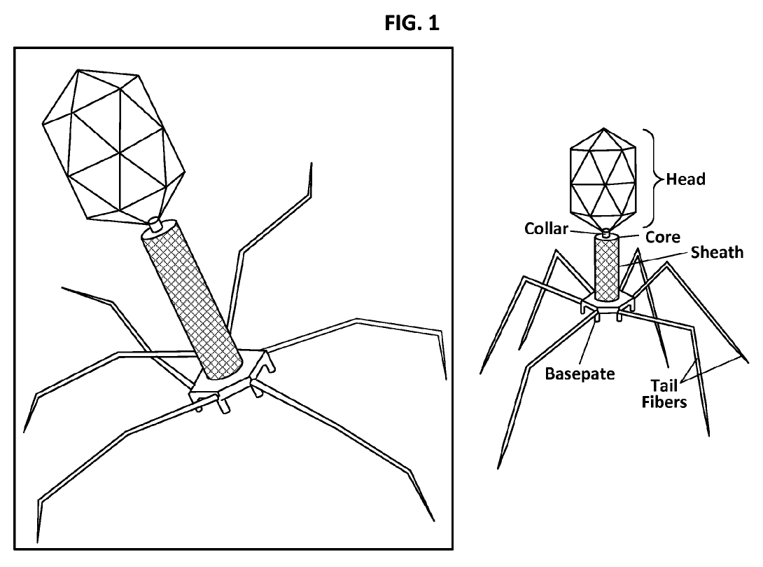

Several varieties of phage exist, and each typically attacks only one species/strain of bacteria. The structure of a bacteriophage is depicted in FIG. 1. Infecting phage attach themselves to the cell wall of the bacterium and inject their genetic material (e.g., a charge of DNA) into the cytoplasm of the bacterium. During the lysogenic phase of phage infection, the phage DNA incorporates into the chromosome of the bacterium and becomes dormant for many generations. At least one environmental inducer is required to cause the phage DNA to excise from the bacterial chromosome and establish the second type of infection, the lytic phase. In this phase, the bacterium is transformed into a phage-making factory. Hundreds of phages are produced and the bacterial cell is lysed to release them. The released phage then find another host bacterium, and the process repeats.

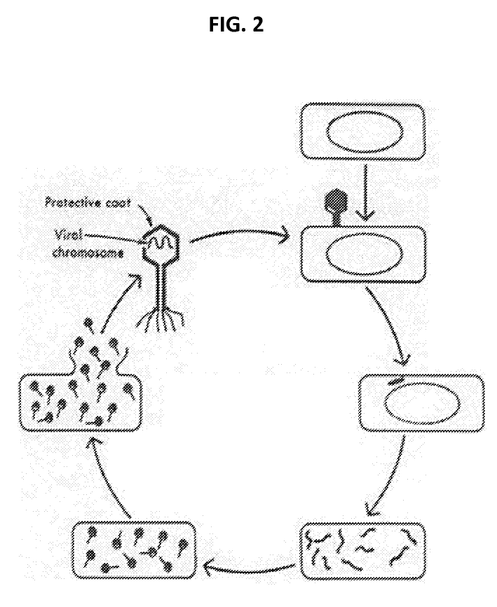

FIG. 2 is a graphic of the replication of a bacteriophage during the lytic cycle. Before viral infection, the cell is involved in replication of its own DNA and transcription and translation of its own genetic information to carry out biosynthesis, growth and cell division. After infection, the viral DNA takes over the machinery of the host cell and uses it to produce the nucleic acids and proteins needed for production of new virus particles. Viral DNA replaces the host cell DNA as a template for both replication (to produce more viral DNA) and transcription (to produce viral mRNA). Viral mRNAs are then translated, using host cell ribosomes, tRNAs and amino acids, into viral proteins such as the coat or tail proteins. The process of DNA replication, synthesis of proteins, and viral assembly is a carefully coordinated and timed event.

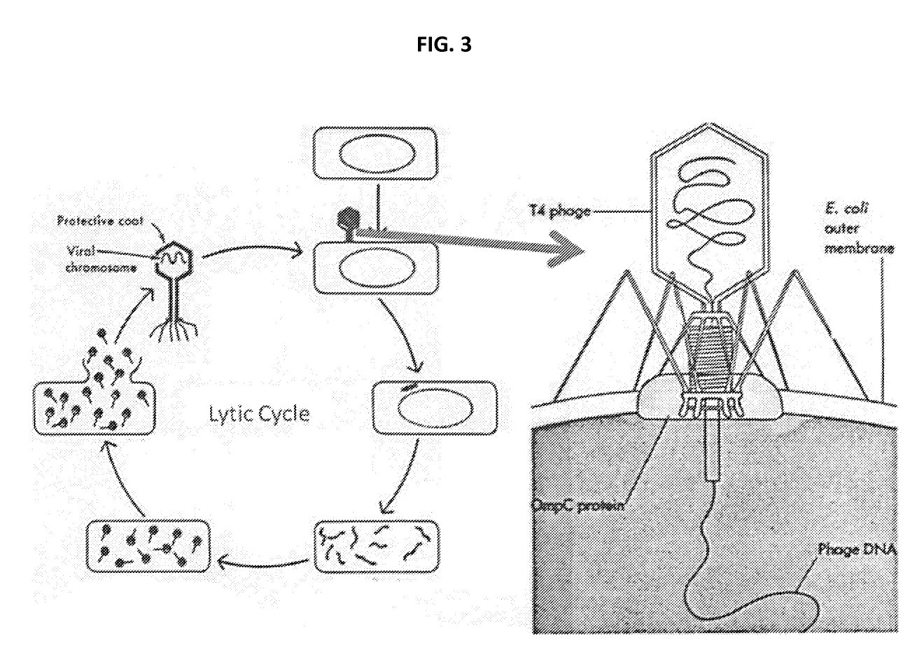

FIG. 3 is a graphic representation of a more complete stage of replication during the lytic cycle. Many bacteriophage that have been studied infect E. coli. The first step in the replication of the phage in its host cell is called adsorption. The phage particle undergoes a chance collision at a chemically complementary site receptors on the bacterial surface, and then adheres to that site by means of its tail fibers.

Following adsorption, the phage injects its DNA (and rarely RNA) into the bacterial cell. The tail sheath contracts and the core is driven through the wall to the membrane. This process is called penetration, and it may be both mechanical and enzymatic. Phage T4 packages a bit of lysozyme in the base of its tail from a previous infection and then uses the lysozyme to degrade a portion of the bacterial cell wall for insertion of the tail core. The DNA is injected into the periplasm of the bacterium; generally it is not known how the DNA penetrates the membrane.

Immediately after injection of the viral DNA, the process called “synthesis of early proteins” is initiated. This refers to the transcription and translation of a section of the phage DNA to make a set of proteins that are needed to replicate the phage DNA. Among the early proteins produced are a repair enzyme to repair the hole in the bacterial cell wall, a DNAase enzyme that degrades the host DNA into precursors of phage DNA, and a virus specific DNA polymerase that will copy and replicate phage DNA. During this period, the cell's energy-generating and protein-synthesizing abilities are maintained, but they are subverted by the virus. The result is the synthesis of several copies of the phage DNA.

The next step is the synthesis of late proteins. Each of the several replicated copies of the phage DNA can be used for transcription and translation of a second set of proteins called the late proteins. The late proteins are mainly structural proteins that make up the capsomeres and the various components of the head and tail assembly. Lysozyme is another late protein that will be packaged in the tail of the phage and used to escape from the host cell during the last step of the replication process.

The replication of phage parts is followed by an assembly process. The proteins that make up the capsomeres assemble themselves into the heads and “reel in” a copy of the phage DNA. The tail and accessory structures assemble and incorporate a bit of lysozyme in the tail plate. The viruses arrange their escape from the host cell during the assembly process.

While the viruses are assembling, lysozyme is being produced as a late viral protein. Some of this lysozyme is used to escape from the host cell by lysing the cell wall peptidoglycan from the inside. This accomplishes the release of the mature viruses, which spread to nearby cells, infect them, and complete additional cycles. The life cycle of a T-phage takes about 25-35 minutes to complete. Because the host cells are ultimately killed by lysis, this type of viral infection is referred to as lytic infection.

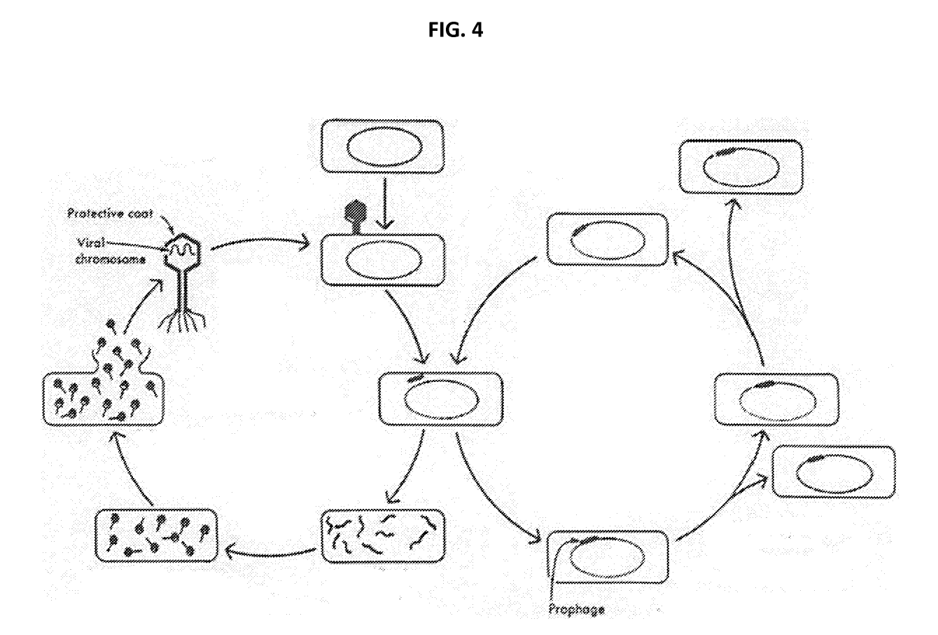

FIG. 4 is a graphic representation of a complete lysogenic cycle. Lysogenic (or “temperate”) infection rarely results in lysis of the bacterial host cell. Lysogenic viruses (e.g., lambda, which infects E. coli) have a different strategy than lytic viruses for their replication. After penetration, the virus DNA integrates into a specific section of the bacterial chromosome and is replicated every time the cell duplicates its chromosomal DNA during normal cell division. Such phage DNA is called “prophage,” and the host bacteria are said to be lysogenized. In the prophage state, all the phage genes except one are repressed, and none of the usual early proteins or structural proteins are produced.

The one phage gene that is expressed codes for the synthesis of a repressor molecule that prevents the synthesis of phage enzymes and proteins required for the lytic cycle. If the synthesis of the repressor molecule stops or if the repressor becomes inactivated, another enzyme encoded by the prophage is synthesized, and the enzyme then excises the viral DNA from the bacterial chromosome. The excised DNA (the phage genome) can then behave like a lytic virus to produce new viral particles and eventually lyse the host cell. This spontaneous derepression is a rare event, occurring about one in 10,000 divisions of a lysogenic bacterium, but it assures that new phage are formed that can proceed to infect other cells.

It can be difficult to recognize lysogenic bacteria, because lysogenic and nonlysogenic cells appear identical. In a few situations, however, the prophage supplies genetic information such that the lysogenic bacteria exhibit a new characteristic (new phenotype) that is not displayed by the nonlysogenic cell. This phenomenon is called lysogenic conversion.

In lytic systems, a protein known as holin is responsible for forming a pore in the cell membrane, such that lysin proteins can target bonds in the peptidoglycan of the cell wall that are necessary component for the wall to remain intact. Lysin thus produces holes in the cell wall peptidoglycan, and the cell membrane is externalized after internal pressure forces it through the hole in the cell wall. This leads to rupture of the membrane and loss of intercellular components, causing cell death. External lysin therapy works only on Gram+ cells, however. Gram− cells have an outer membrane covering the peptidoglycan cell wall, so lysin is not able to form a hole without a holin to degrade the cell membrane.

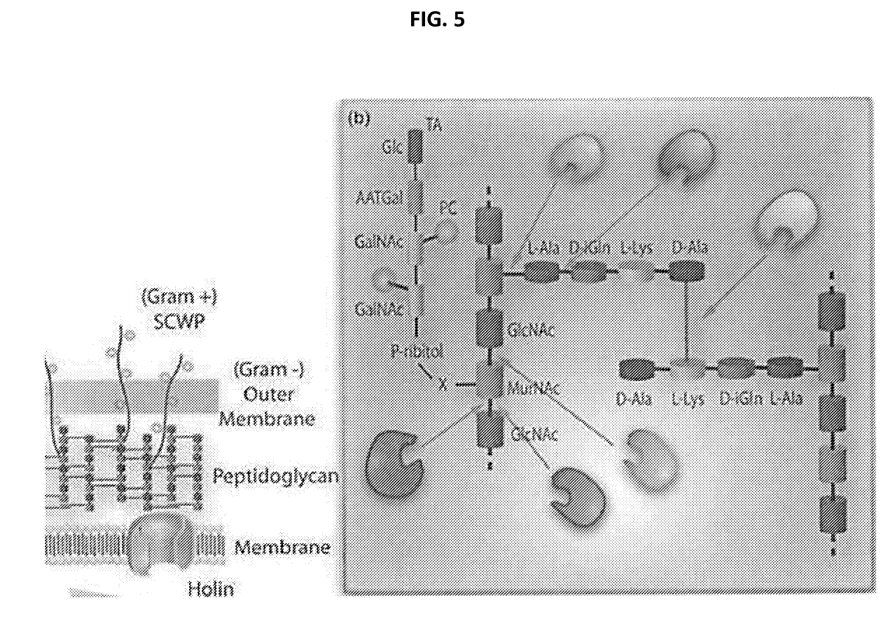

FIG. 5 shows a graphic representation of a phage lysin system. When a phage is inside a bacterial cell, it needs to produce holins in order for the lysins to reach the cell wall peptidoglycan. Holins are small membrane proteins that accumulate in the membrane until, at a specific time that is “programmed” into the holin gene, the membrane suddenly becomes permeabilized to the fully folded endolysin. Destruction of the murein bacterial cell wall and bursting of the cell are immediate sequelae. Holins control the length of the infective cycle for lytic phages, and thus are subject to intense evolutionary pressure to achieve lysis at an optimal time. Holins are regulated protein inhibitors of several different kinds. Each of the different circled enzymes in FIG. 5 represents a different type of lysin that is specific to a different bond within the peptidoglycan. Cleavage of any one of these bonds can degrade the cell wall. When lysin is introduced from the external environment, a holin is not required but is optional.

As described herein, a novel phage lysin was isolated and characterized, and shown to be active against P. larvae in culture. Thus, this document provides methods for lysing P. larvae cells by contacting them with a lysin polypeptide. The methods provided herein can include, for example, providing to an environment of a bee hive infected with P. larvae an isolated lysin polypeptide or a nucleic acid encoding the lysin polypeptide. In some embodiments, the lysin polypeptide or nucleic acid can be contained within a composition, and can be provided directly to bee larvae (e.g., in larvae food, or in another composition that larvae can ingest) or can be applied to the bee hive or portions thereof. Lysing of the P. larvae can cause a hole in the peptidoglycan of the cell wall, and the cell membrane of the P. larvae can be is externalized due to internal pressure that forces the membrane through the hole in the cell wall, leading to rupture of the cell membrane and loss of intercellular components. Rupture of the cell membrane can lead to death of the P. larvae cells. The methods provided herein therefore can be used to treat or prevent AFB in a population of honeybees.

The lysin polypeptide can contain the amino acid sequence set forth in SEQ ID NO:2 herein. In some embodiments, the lysin polypeptide can include an amino acid sequence that is at least 90 percent (e.g., at least 91, at least 92, at least 93, at least 94, at least 95, at least 96, at least 97, at least 98, or at least 99 percent) identical to the sequence set forth in SEQ ID NO:2.

The percent sequence identity between a particular nucleic acid or amino acid sequence and a sequence referenced by a particular sequence identification number is determined as follows. First, a nucleic acid or amino acid sequence is compared to the sequence set forth in a particular sequence identification number using the BLAST 2 Sequences (Bl2seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the Bl2seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default setting. For example, the following command can be used to generate an output file containing a comparison between two sequences: C:\Bl2seq c:\seq1.txt -j c:\seq2.txt -p blastn -o c:\output.txt -q −1 -r 2. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence (e.g., SEQ ID NO:2), or by an articulated length (e.g., 100 consecutive nucleotides or amino acid residues from a sequence set forth in an identified sequence), followed by multiplying the resulting value by 100. For example, a nucleic acid sequence that has 210 matches when aligned with the sequence set forth in SEQ ID NO:2 is 93.8 percent identical to the sequence set forth in SEQ ID NO:2 (i.e., 210÷224×100=93.8). It is noted that the percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. It also is noted that the length value will always be an integer.

This document also provides isolated nucleic acids having a sequence with at least 90 percent but less than 100 percent sequence identity to the nucleotide sequence set forth in SEQ ID NO:1, and purified polypeptides having a sequence with at least 90 percent but less than 100 percent sequence identity to the amino acid sequence set forth in SEQ ID NO:2. For example, a polypeptide can have one or more additions subtractions, or substitutions as compared to SEQ ID NO:2. Polypeptides having one or more amino acid substitutions relative to SEQ ID NO:2 can be prepared using methods known in the art.

In some embodiments, amino acid substitutions can be conservative amino acid substitutions. In some cases, amino acid substitutions can be substitutions that do not differ significantly in their effect on maintaining (a) the structure of the peptide backbone in the area of the substitution, (b) the charge or hydrophobicity of the molecule at the target site, or (c) the bulk of the side chain. Naturally occurring residues are divided into groups based on common side-chain properties: (1) hydrophobic: norleucine, methionine, alanine, valine, leucine, isoleucine; (2) neutral hydrophilic: cysteine, serine, threonine; (3) acidic: aspartate, glutamate; (4) basic: asparagine, glutamine, histidine, lysine, arginine; (5) residues that influence chain orientation: glycine, proline; and (6) aromatic; tryptophan, tyrosine, phenylalanine. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. It is reasonable to expect that replacement of a leucine with an isoleucine or valine, an aspartate with a glutamate, a threonine with a serine, or a similar replacement of an amino acid with a structurally related amino acid will not have a major effect on the properties of the resulting polypeptide. In some cases, non-conservative substitutions can be used. A non-conservative substitution can include exchanging a member of one of the classes described herein for another. Whether an amino acid change results in a functional polypeptide can readily be determined by assaying the specific activity of the polypeptide (e.g., using a method for assessing lytic activity against P. larvae, as described herein).

As used herein, “isolated,” when in reference to a nucleic acid, refers to a nucleic acid that is separated from other nucleic acids that are present in a genome, e.g., a plant genome, including nucleic acids that normally flank one or both sides of the nucleic acid in the genome. The term “isolated” as used herein with respect to nucleic acids also includes any non-naturally-occurring sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences, as well as DNA that is incorporated into a vector, an autonomously replicating plasmid, a virus (e.g., a pararetrovirus, a retrovirus, lentivirus, adenovirus, or herpes virus), or the genomic DNA of a prokaryote or eukaryote. In addition, an isolated nucleic acid can include a recombinant nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

A nucleic acid can be made by, for example, chemical synthesis or polymerase chain reaction (PCR). PCR can be used to amplify specific sequences from DNA as well as RNA, including sequences from total genomic DNA or total cellular RNA. Various PCR methods are described, for example, in PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

Isolated nucleic acids also can be obtained by mutagenesis. For example, a donor nucleic acid sequence can be mutated using standard techniques, including oligonucleotide-directed mutagenesis and site-directed mutagenesis through PCR. See, Short Protocols in Molecular Biology, Chapter 8, Green Publishing Associates and John Wiley & Sons, edited by Ausubel et al., 1992.

The term “polypeptide” as used herein refers to a compound of two or more subunit amino acids regardless of post-translational modification (e.g., phosphorylation or glycosylation). The subunits may be linked by peptide bonds or other bonds such as, for example, ester or ether bonds. The term “amino acid” is intended to include natural and unnatural or synthetic amino acids, including D/L optical isomers.

By “isolated” or “purified” with respect to a polypeptide it is meant that the polypeptide is separated to some extent from the cellular components with which it is normally found in nature (e.g., other polypeptides, lipids, carbohydrates, and nucleic acids). A purified polypeptide can yield a single major band on a non-reducing polyacrylamide gel. A purified polypeptide can be at least about 75% pure (e.g., at least 80%, 85%, 90%, 95%, 97%, 98%, 99%, or 100% pure). Purified polypeptides can be obtained by, for example, extraction from a natural source, by chemical synthesis, or by recombinant production in a host cell or transgenic plant, and can be purified using, for example, affinity chromatography, immunoprecipitation, size exclusion chromatography, and ion exchange chromatography. The extent of purification can be measured using any appropriate method, including, without limitation, column chromatography, polyacrylamide gel electrophoresis, or high-performance liquid chromatography.

Recombinant nucleic acid constructs (e.g., vectors) containing lysin nucleic acids also are provided herein. A “vector” is a replicon, such as a plasmid, phage, or cosmid, into which another DNA segment may be inserted so as to bring about the replication of the inserted segment. Generally, a vector is capable of replication when associated with the proper control elements. Suitable vector backbones include, for example, those routinely used in the art such as plasmids, viruses, artificial chromosomes, BACs, YACs, or PACs. The term “vector” includes cloning and expression vectors, as well as viral vectors and integrating vectors. An “expression vector” is a vector that includes one or more expression control sequences, and an “expression control sequence” is a DNA sequence that controls and regulates the transcription and/or translation of another DNA sequence. Suitable expression vectors include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

In a construct as provided herein, a lysin coding sequence can be operably linked to a regulatory region that controls expression of the coding sequence. The terms “regulatory region,” “control element,” and “expression control sequence” refer to nucleotide sequences that influence transcription or translation initiation and rate, and stability and/or mobility of the transcript or polypeptide product. Regulatory regions include, without limitation, promoter sequences, enhancer sequences, response elements, protein recognition sites, inducible elements, promoter control elements, protein binding sequences, 5′ and 3′ untranslated regions (UTRs), transcriptional start sites, termination sequences, polyadenylation sequences, introns, and other regulatory regions that can reside within coding sequences, such as secretory signals, Nuclear Localization Sequences (NLS) and protease cleavage sites.

As used herein, “operably linked” means incorporated into a genetic construct so that expression control sequences effectively control expression of a coding sequence of interest. A coding sequence is “operably linked” and “under the control” of expression control sequences in a cell when RNA polymerase is able to transcribe the coding sequence into RNA, which if an mRNA, then can be translated into the protein encoded by the coding sequence. Thus, a regulatory region can modulate, e.g., regulate, facilitate or drive, transcription in the plant cell, plant, or plant tissue in which it is desired to express a modified target nucleic acid.

The vectors provided herein also can include, for example, origins of replication, and/or scaffold attachment regions (SARs). In addition, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

The methods provided herein can be used to treat, prevent, or reduce the risk of a P. larvae infection in a honeybee or a population of honeybees. In some embodiments, the methods provided herein can include administering to a honeybee a composition containing a lysin polypeptide that is capable of lysing more than one strain of P. larvae. Any suitable amount of polypeptide or nucleic acid can be administered. in some embodiments, for example, a method can include administering at least 100 mg (e.g., at least 100 mg, at least 500 mg, at least 1 g, at least 2.5 g, at least 5 g, or more than 5 g) of a lysin polypeptide to the environment of a bee hive. The terms “treatment” and “treating” refer to an intervention (e.g., the administration of an agent to a subject) that prevents, slows, or delays the onset or progression of a disease or reduces (e.g., eradicates) its incidence within a treated subject (e.g., a honey bee).

The methods provided herein can include, for example, providing to an environment of a bee hive infected with P. larvae an isolated lysin polypeptide or a nucleic acid encoding the lysin polypeptide. In some embodiments, the lysin polypeptide or nucleic acid can be contained within a composition, and can be provided directly to bee larvae (e.g., in larvae food, or in another composition that larvae can ingest) or can be applied to the bee hive or portions thereof. In some cases, the method can include providing a population of honeybees (e.g., a population that includes larvae) with a composition (e.g., a nutritional medium such as worker jelly or royal jelly) containing an amount of indole, phenol, or a derivative thereof, that is effective to prevent germination of P. larvae spores. Nutritional media for honeybees can be obtained commercially, for example, and supplemented with a compound as described herein.

The invention will be further described in the following example, which does not limit the scope of the invention described in the claims.

### EXAMPLE

### Isolation and Characterization of a Novel Phage Lysin Active Against P. larvae

During the lytic cycle of viral infection, bacteriophages express proteins called lysins after genome replication and capsid assembly. A holin protein is expressed first, and creates lesions in the inner bacterial membrane. The lysin can then traverse these lesions and hydrolyze peptidoglycan, causing lysis of the cell and release of progeny virions, which completes the viral life cycle. In Gram-positive bacteria, the peptidoglycan layer is exposed, so exogenous application of purified recombinant lysin can cause rapid cell death even without the holin or viral infection. Lysins are modular, possessing one or more conserved N-terminal catalytic domains and one C-terminal cell wall binding domain. Using bioinformatics, the genomes of numerous P. larvae phage were analyzed, revealing extensive conservation of lysin and holin genes. A bacteriophage lysin (PlyPalA) was identified from the genome of a novel bacteriophage that infects P. larvae. PlyPalA has an N-acetylmuramoyl-L-alanine amidase N-terminal domain, and it caused significant reduction in turbidity of pathogenic log-phase P. larvae cultures. PlyPalA also had a wide pH range of activity, and was readily expressed in transformed E. coli and purified by ion exchange chromatography.

Materials and methods: Genomic phage DNA was extracted and then sequenced using Illumina NextGen sequencing, followed by annotation and identification of putative lysin genes in DNA Master. Comparative genomics using previously-sequenced P. larvae phages was carried out using Mauve. PlyPalA was identified with BLAST due to high similarity to known phage lysins. Primers specific for the lysin gene plypalA were engineered with PstI and EcoRI restriction sites, ligated into the plasmid pBAD24, and cloned into E. coli strain MON1. Positive transformants were screened with a soft agar overlay containing P. larvae and identified by a distinct clearing around the colonies.

A positive transformant was cultured in LB supplemented with ampicillin, and expression of PlyPalA was induced with arabinose. Total proteins were extracted by chloroform and bead beating. The lysin, PlyPalA, was purified by salting out with 40% saturation of (NH₄)₂SO₄, cation exchange chromatography (CEC), and anion exchange chromatography (AEC). Purity was verified by SDS-PAGE. The lysin was quantified with a bicinchoninic acid (BCA) assay in which reduced copper ions form a purple-colored complex in the presence of protein. Lysis assays were carried out in triplicate on 96-well plates at 35° C. using a Tecan plate reader.

The nucleic acid sequence of PlyPalA is:

(SEQ ID NO: 1)
ATGATGGAAATCAGAGAAATGCTAGTAGACCCAAGTAAATATGGAATTA
AATGTCCGAATAAAATGGCACCGAAATATATTACGTTCCACAATACGTAT
AACGATGCTCCCGCAGAAAATGAGGTTCGTTACATGATCGGGAACAATAA
CGAGGTTTCGTTCCACGTTGCTGTGGATGATAAGGAAGCTGTTCAGGGCA
TTCCTTTTGATCGAAATGCCTGGCATTGTGGAGACGGGAACGGGACAGGA
AACCGTCAATCCATCGGCGTAGAGATTTGTTATTCCAAGTCCGGCGGTAA
CCGATATTATAAGGCCGAGGACAATGCGGCTATTATCATTGCCCAGCTTA
TGAAACAGTTTTGTATTCCTATTGAGAATGTGGTTCCACACCAGCACTGG
AGTGGTAAATACTGTCCGCACAGAATGTTAGATGAGGGAAGAGTGCCAAG
CTTTATAGAGCGAATTAAACAAGCATACGAAGGAGAGGAAGACGACATGA
GTAGAACATTACAACTGGAAGATTGGCAATGGAAACAGCTCTATGACAAT
ATGGGGAAAGTCTGGAATGCAGGGAAATTTACAGACTGGAATTGGATGGT
TAAAATAGAAAACCGTTGCCTTACCGTTGATGAGCTGGCATGGCTTAACA
ACCACATTTTAGCGAGTAGCCTGTAG

The amino acid sequence for the encoding protein is:

MMEIREMLVDPSKYGIKCPNKMAPKYITFHNTYNDAPAENEVRYMIGNN NEVSFHVAVDDKEAVQGIPFDRNAWHCGDGNGTGNRQSIGVEICYSKSGGNRYY KAEDNAAIIIAQLMKQFCIPIENVVPHQHWSGKYCPHRMLDEGRVPSFIERIKQAY EGEEDDMSRTLQLEDWQWKQLYDNMGKVWNAGKFTDWNWMVKIENRCLTVD ELAWLNNHILASSL (SEQ ID NO:2)

A hybrid method for phage DNA extraction: Phage capsids were disrupted by Proteinase K and SDS. Phenol was added to denature proteins and chloroform was used to remove phenol, directly yielding intact genomes suitable for sequencing and PCR. After sequencing, they were annotated and putative lysins and holins were identified and analyzed bioinformatically.

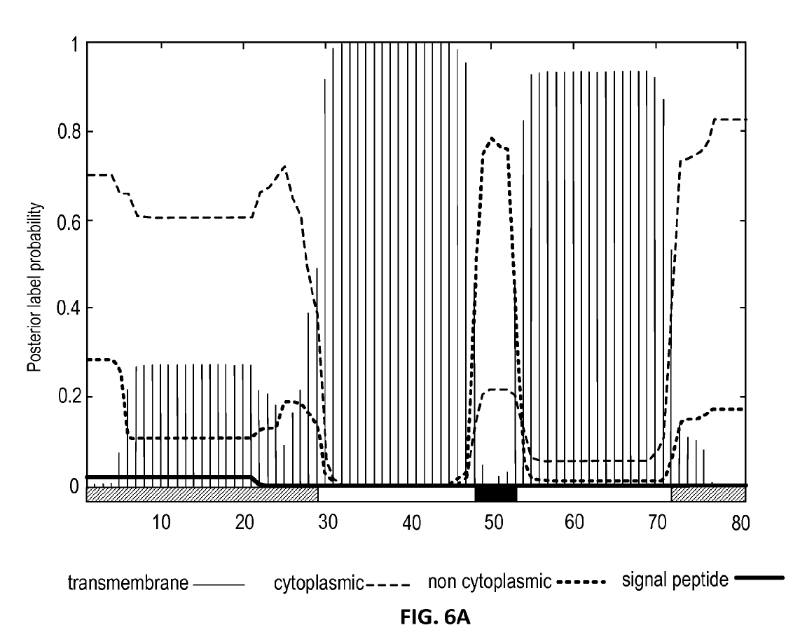

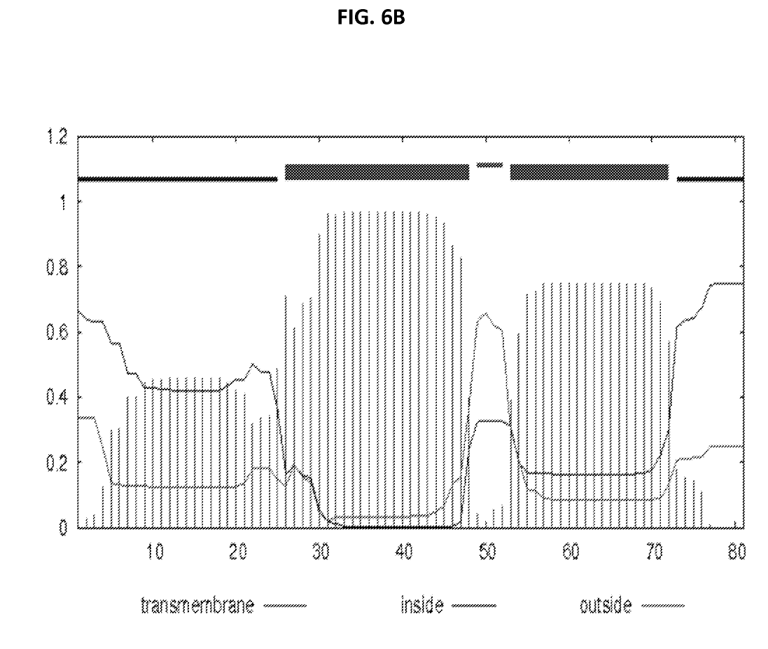

The gene for PlyPalA is immediately upstream of a gene for a putative phage holin: Phobius (FIG. 6A) and TMHMM (FIG. 6B) both predicted two transmembrane domains and cytoplasmic termini for XIII_gp38, strongly suggesting that it is a class II phage holin.

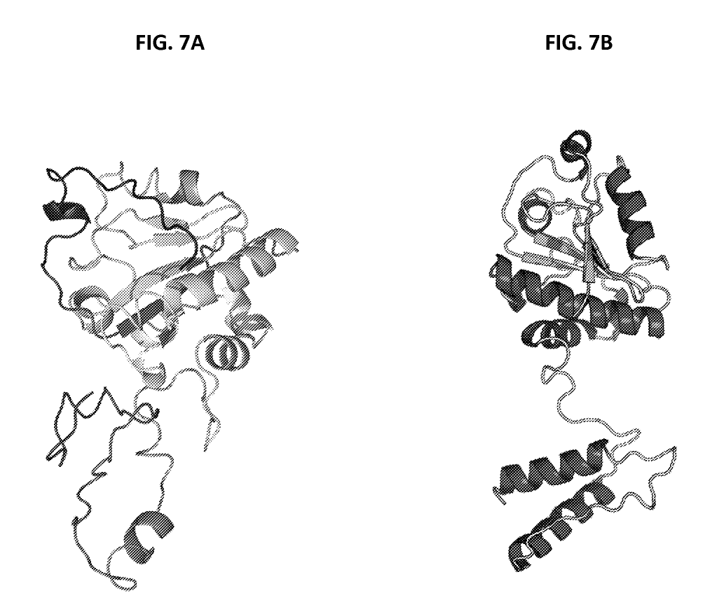

Predicted 3D structure of PlyPalA: Phyre2 (FIG. 7A) and RaptorX (FIG. 7B) use a combination of homology and ab initio modeling to predict the tertiary structure of PlyPalA from its primary sequence. The N-terminus is depicted on top and the C-terminus is depicted at the bottom of FIGS. 7A and 7B.

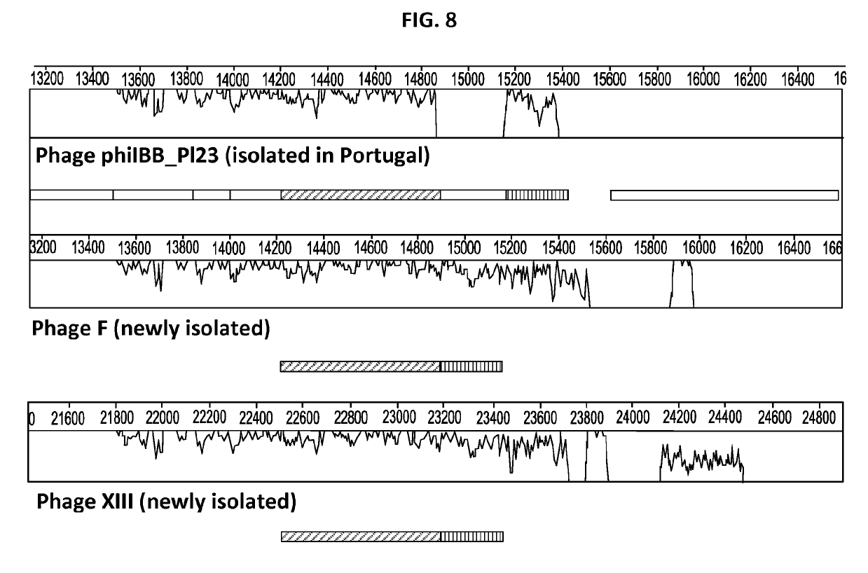

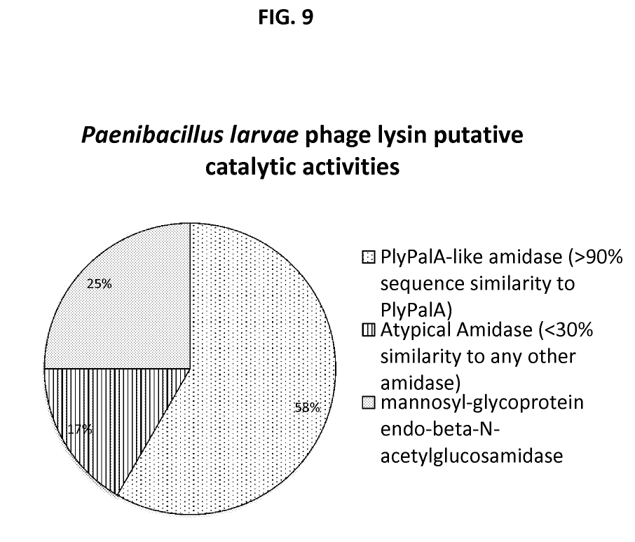

Comparative genomics of P. larvae phage lysis cassettes: A comparative analysis of three phage genomes is shown in FIG. 8. Hatched bars indicate homologous lysin genes and vertically striped bars indicate homologous holin genes. Regions with lighter shading indicate conservation among all 3 genomes, while regions with darker shading indicate conservation among 2 of 3 genomes. Annotated P. larvae phages (newly annotated and from GENBANK®) were scanned for lysins, and the sequences were compared to assess putative catalytic activities. As depicted in FIG. 9, 58% were predicted to have PlyPalA-like amidase activity (with greater than 90% sequence similarity to PlyPalA), 25% were predicted to have mannosyl-glycoprotein endo-beta-N-acetylglucosamidase activity, and 17% were predicted to have atypical amidase activity (with less than 30% sequence similarity to any other amidase).

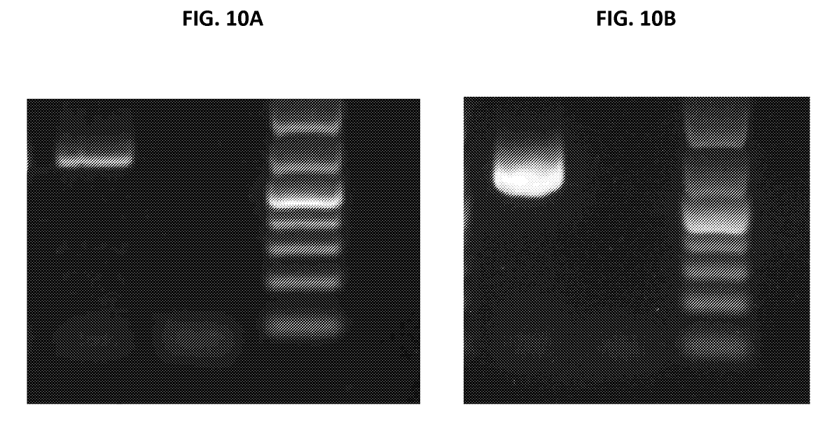

Phusion PCR amplification of lysin genes: Phusion is a DNA polymerase with an error rate of 4.4×10⁻⁷, approximately 50 times less than that of Taq polymerase. Phusion was used to amplify plypalA from phage XIII (FIG. 10A) and plypalB from phage F (FIG. 10B), genes whose products are closely related amidases.

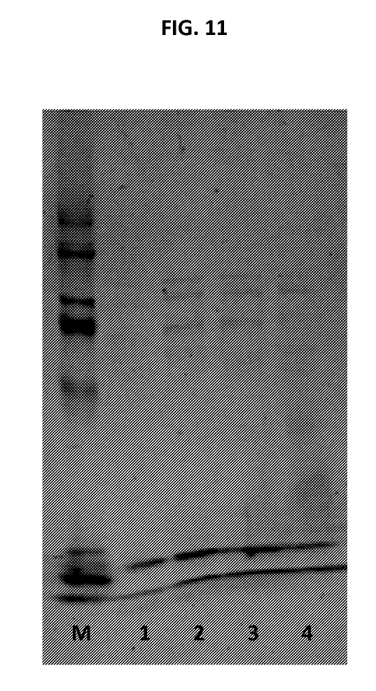

SDS-PAGE of the progressive purification of PlyPalA: A 7.5% acrylamide gel showing purification is shown in FIG. 11. Lanes from left to right: M, 10-250 kDa ladder from New England Biolabs; 1, AEC (DEAE) 600 mM NaCl eluate; 2, CEC (CM) flowthrough; 3, dialysate after salting out; 4, crude E. coli lysate. The presence of two bands likely was due to re-oxidation of cysteine residues during the run, forming disulfide bridges that affect how the protein migrated through the gel.

Lytic activity of PlyPalA against P. larvae strains: P. larvae strains were grown to log-phase, washed in PBS, and frozen at −80° C. Cells were resuspended in buffer, and 180 μL of the cell suspension was combined with either 20 μL of purified lysin or buffer, in triplicate. Effective % drop in OD600 was calculated by subtracting the buffer control % drop from the treated % drop after 30 minutes of incubation at 35° C. E. coli was used as a negative control. Results are summarized in Table 1.

TABLE 1

| | Effective |
|---|---|
| Bacterial Strain | % Drop in OD₆₀₀ |
| P. larvae 16425 | 68.77 |
| P. larvae 2188 | 65.88 |
| P. larvae 748 | 54.10 |
| P. larvae 3544 | 48.45 |
| P. larvae 2231 | 45.39 |
| P. larvae 747 | 43.76 |
| P. larvae 2605 | 38.04 |
| P. larvae 3688 | 30.23 |
| P. larvae 367 | 18.73 |
| P. larvae 843 | 11.84 |
| P. larvae 368 | 7.36 |
| E. coli 11303B | 0.28 |

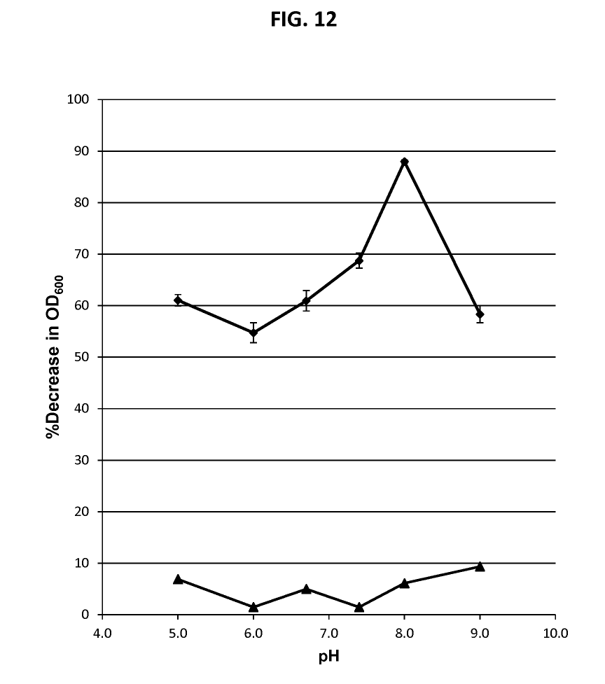

pH activity range of PlyPalA: P. larvae strain 748 was grown to log-phase, and 270 μL was treated with 30 μL of purified lysin or buffer. Percentage drop in OD₆₀₀ was calculated after 67 minutes of incubation at 35° C., and is plotted on FIG. 12 (upper line). Error bars indicate standard deviations for triplicates. The lower line indicates drop in OD₆₀₀ for buffer controls.

Further experiments: The genomes of XIII and F appear to have assembled into one contig with high coverage and seem complete; sequencing reads of WA's genome are assembled. Additionally, PlyPalB, which has a high (<90%) amino acid similarity to PlyPalA, is expressed and purified to test whether its range of activity is different from PlyPalA. SDS-PAGE is conducted with additional reducing agent to verify that the lytic activity indeed comes from one protein.

In addition, the specificity of PlyPalA in vitro is further characterized. In particular, its activity against Paenibacillus spp., as well as its activity against other genera of Gram-positive bacteria, such as Lactobacillus spp., is evaluated to ensure that it will not harm commensal organisms in the honey bee larva microbiome. Its stability in heat and its ability to be lyophilized are determined. Once in vitro tests are completed, PlyPalA and the other lysins are tested in vivo, in honey bee larvae that are raised in the lab and infected with P. larvae endospores.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for killing a Paenibacillus larvae cell, comprising contacting the cell with a lysin polypeptide having an amino acid sequence with at least 90 percent sequence identity to SEQ ID NO:2.

2. The method of claim 1, wherein the P larvae cell is in a honeybee larva.

3. The method of claim 1, wherein said contacting comprises providing the lysin protein to the environment of a honeybee hive.

4. The method of claim 1, wherein the lysin polypeptide has an amino acid sequence with at least 95 percent sequence identity to SEQ ID NO:2.

5. A purified polypeptide having an amino acid sequence that is at least 90 percent but less than 100 percent identical to the sequence set forth in SEQ ID NO:2.

6. The purified polypeptide of claim 5, wherein the amino acid sequence is at least 95 percent but less than 100 percent identical to the sequence set forth in SEQ ID NO:2.

7. A composition comprising the polypeptide of claim 5.

8. The composition of claim 7, wherein the composition comprises honeybee food.

9. (canceled)

10. (canceled)

11. A method for treating American foulbrood in a population of honeybees, comprising administering to the population a lysin polypeptide having an amino acid sequence with at least 90 percent sequence identity to SEQ ID NO:2.

12. The method of claim 11, wherein the population comprises honeybee larvae.

13. The method of claim 11, wherein said administering comprises providing the lysin protein to the environment of a honeybee hive.

14. The method of claim 11, wherein said administering comprises providing a composition that comprises the lysin protein to the environment of a honeybee hive.

15. The method of claim 11, wherein said administering comprises providing a nucleic acid encoding the lysin protein to the environment of a honeybee hive.

16. The method of claim 11, wherein the lysin polypeptide has an amino acid sequence with at least 95 percent sequence identity to SEQ ID NO:2.
